(19) United States
(12) Patent Application Publication
(10) Pub. No.: US 2026/0244938 A1
(43) Pub. Date: Aug. 20, 2026

(54) ACTIVITY PREDICTION

(71) Applicant: OK2STANDUP, INC., Bedford, PA (US)

(72) Inventors: Eunice Eun Young YANG, Bedford, PA (US); Daniel D. WARFIELD, Bedford, PA (US)

(21) Appl. No.: 19/472,288

(22) PCT Filed: Mar. 29, 2024
(86) PCT No.: PCT/US2024/022141

§ 371 Date: Oct. 3, 2025

Related U.S. Application Data
(60) Provisional application No. 63/494,382, filed on Apr. 5, 2023.

(51) Int. Cl. G06N 3/09; G06N 3/044; G06N 3/0464

(52) U.S. Cl. CPC G06N 3/09; G06N 3/044; G06N 3/0464

(57) ABSTRACT

Systems and techniques for training an activity prediction model and for performing activity prediction utilizing the activity prediction model are described herein. An activity prediction model may be trained by training potential activity prediction models using machine learning based on an input dataset including activity of an individual from a training phase, ground truth dataset and different hyperparameters, performing hyperparameter optimization, and optimizing the potential activity prediction models for accuracy or minimizing loss. Thereafter, the activity prediction model may be utilized by passing an execution dataset including a set of sensor data associated with activity of an individual from an execution phase to generate an activity prediction which may be indicative of a current or future activity of the individual.

### BACKGROUND

Each year, millions of older people fall. Statistics have shown that approximately one out of four older people fall each year. Further, falling once doubles an individual's chances of falling again. Falls may cause injuries, such as broken bones and may result in a trip to an emergency room or otherwise require hospitalization. Many different conditions may contribute to falling and these conditions are called risk factors. Risk factors may include lower body weakness, vitamin D deficiency, lack of coordination, use of medicines, such as tranquilizers, sedatives, or antidepressants, vision problems, foot pain or poor footwear, hazards such as broken or uneven steps, or obstacles, such as rugs that may be tripped over.

### BRIEF DESCRIPTION

A model generator may generate an activity prediction model which may be a function y=f(x) which maps an input x to an output y. During an execution phase, the activity prediction model may generate the output y which may be an activity prediction based on sensor data x associated with activity of an individual. The activity prediction y may be one of two or more activity prediction states, such as an inactive activity state, a sitting activity state, or an active activity state. According to one aspect, a window of predetermined time steps of sensor data may be utilized as the input x to the activity prediction model f. For example, this input x to the activity prediction model f may include accelerometer data associated with activity of an individual over the time window.

The activity prediction model may be trained by the model generator during a training phase, such as by training potential activity prediction models using machine learning based on an input dataset or ground truth dataset and different hyperparameters, performing hyperparameter optimization, and optimizing the potential activity prediction models for accuracy or minimizing loss.

According to one aspect, a system for training an activity prediction model may include a memory and a processor. The memory may store one or more instructions. The processor may execute one or more of the instructions stored on the memory to perform one or more acts, actions, or steps. For example, the processor may perform receiving an input dataset. The input dataset may include a set of sensor data associated with activity of an individual from the training phase and a set of flags associated with the set of sensor data. One or more of the flags from the set of flags may be indicative of an onset of a transition from a first activity state to a second activity state for the individual from the training phase. The processor may perform training two or more potential activity prediction models using machine learning based on a first subset of the input dataset and one or more hyperparameters, optimizing one or more of the hyperparameters of the two or more potential activity prediction models based on a second subset of the input dataset, and selecting one of the two or more potential activity prediction models as an activity prediction model based on a third subset of the input dataset. The activity prediction model may thereby be utilized to generate an activity prediction based on an execution dataset including a second set of sensor data associated with activity of a second individual during an execution phase.

The set of sensor data may include accelerometer data associated with activity of the individual. The set of flags associated with the set of sensor data may be human annotated or model annotated. The optimizing one or more of the hyperparameters may occur prior to the selecting one of the two or more potential activity prediction models as the activity prediction model. The selecting one of the two or more potential activity prediction models as the activity prediction model may occur prior to the optimizing one or more of the hyperparameters. One or more of the hyperparameters may include a type of neural network, a number of convolutions, a number of layers, or a window size associated with the set of sensor data. The type of neural network may include a convolutional neural network (CNN), a recurrent neural network (RNN), or a long short-term memory (LSTM) neural network. The activity prediction generated by the activity prediction model may be indicative of a current activity of the second individual. The activity prediction generated by the activity prediction model may be indicative of a future activity of the second individual. The training of the two or more potential activity prediction models may be performed using different hyperparameters for each of the two or more potential activity prediction models.

According to one aspect, a system for activity prediction may include a memory and a processor. The memory may store one or more instructions. The processor may execute one or more of the instructions stored on the memory to perform one or more acts, actions, or steps. For example, the processor may perform receiving an execution dataset. The execution dataset may include a set of sensor data associated with activity of an individual from an execution phase. The processor may perform passing the execution dataset through an activity prediction model to generate an activity prediction. The activity prediction model may be trained using machine learning based on a first subset of an input dataset and one or more hyperparameters. The input dataset may include a set of sensor data associated with activity of an individual from a training phase and a set of flags associated with the set of sensor data. One or more of the flags from the set of flags may be indicative of an onset of a transition from a first activity state to a second activity state for the individual from the training phase. One or more of the hyperparameters may be optimized based on a second subset of the input dataset. The activity prediction model may be selected from two or more potential activity prediction models based on a third subset of the input dataset.

The system for activity prediction may include a sensor sensing data of the execution dataset and transmitting the data to the processor. The sensor may be an accelerometer, an image capture device, a heartbeat sensor, a cardiac sensor, a temperature sensor, an electrocardiogram sensor, an oxygen sensor, a blood pressure sensor, or a biometric sensor. One or more of the hyperparameters may include a type of neural network, a number of convolutions, a number of layers, or a window size associated with the set of sensor data. The activity prediction generated by the activity prediction model may be indicative of a current activity of the individual from the execution phase. The activity prediction generated by the activity prediction model may be indicative of a future activity of the individual from the execution phase.

According to one aspect, a computer-implemented method for activity prediction may include sensing, via a sensor, data of an execution dataset and receiving, via a processor, an execution dataset. The execution dataset may include a set of sensor data associated with activity of an individual from an execution phase. The computer-implemented method for activity prediction may include passing, via the processor, the execution dataset through an activity prediction model to generate an activity prediction. The activity prediction model may be trained using machine learning based on a first subset of an input dataset and one or more hyperparameters. The input dataset may include a set of sensor data associated with activity of an individual from a training phase and a set of flags associated with the set of sensor data. One or more of the flags from the set of flags may be indicative of an onset of a transition from a first activity state to a second activity state for the individual from the training phase. One or more of the hyperparameters may be optimized based on a second subset of the input dataset. The activity prediction model may be selected from two or more potential activity prediction models based on a third subset of the input dataset.

The first activity state from the input dataset may be laying down and the second activity state from the input dataset may be a higher activity posture than the first activity state of laying down. The sensor may be an accelerometer, an image capture device, a heartbeat sensor, a cardiac sensor, a temperature sensor, an electrocardiogram sensor, an oxygen sensor, a blood pressure sensor, or a biometric sensor. One or more of the hyperparameters may include a type of neural network, a number of convolutions, a number of layers, or a window size associated with the set of sensor data.

### BRIEF DESCRIPTION OF THE DRAWINGS

FIG. 1 is an exemplary component diagram of a system for training an activity prediction model and/or a system for activity prediction, according to one aspect.

FIG. 2 is an exemplary flow diagram of a method for training an activity prediction model, according to one aspect.

FIG. 3 is an exemplary flow diagram of a method for training an activity prediction model, according to one aspect.

FIG. 4 is an exemplary flow diagram of a method for activity prediction, according to one aspect.

FIG. 5 is an exemplary illustration of data which may be utilized in accordance with any of the systems or methods of FIGS. 1-4, according to one aspect.

FIG. 6 is an exemplary sensor for the system for activity prediction of FIG. 1, according to one aspect.

FIG. 7 is an exemplary application for a mobile device of the system for activity prediction of FIG. 1, according to one aspect.

FIG. 8 is an illustration of an example computer-readable medium or computer-readable device including processor-executable instructions configured to embody one or more of the provisions set forth herein, according to one aspect.

FIG. 9 is an illustration of an example computing environment where one or more of the provisions set forth herein are implemented, according to one aspect.

### DETAILED DESCRIPTION

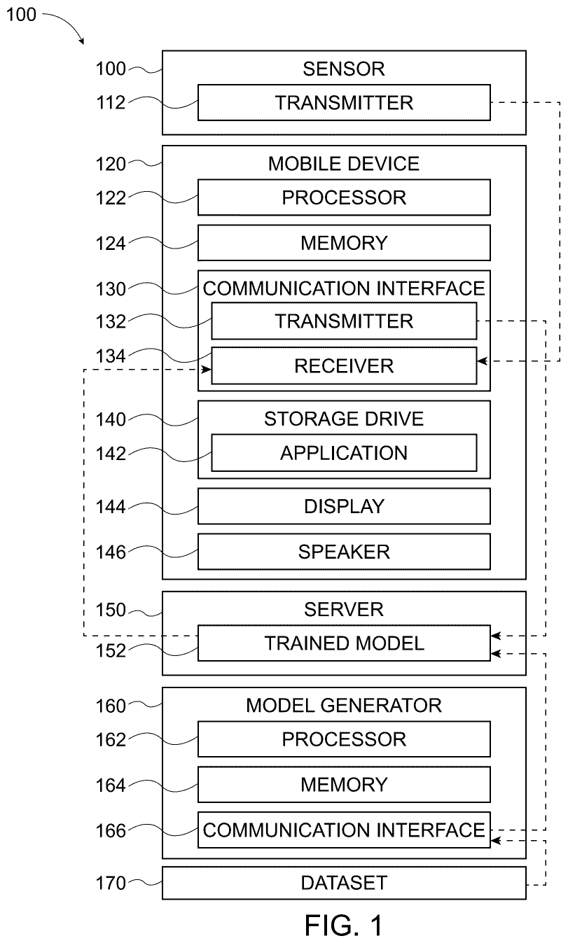

FIG. 1 is an exemplary component diagram of a system for training an activity prediction model and/or a system for activity prediction, according to one aspect. In this regard, it will be appreciated that different portions or configurations of the system 100 of FIG. 1 may be utilized as a system for training an activity prediction model as well as a system for activity prediction which utilizes the activity prediction model.

As a brief introduction to one or more components of the system 100 of FIG. 1, the system 100 may include one or more of a sensor 110 including a transmitter 112, a mobile device 120 including a processor 122, a memory 124, a communication interface 130 having a transmitter 132 and a receiver 134, a storage drive 140 for storing an application 142 or activity prediction application to be executed, a display 144, and a speaker 146. The system 100 may include a server 150 which may house or store the activity prediction model 152, which may be generated by a model generator 160 including a processor 162, a memory 164, and a communication interface 166 based on an input dataset 170. It will be appreciated that components within the mobile device 120 or within the model generator 160 may be communicatively coupled via one or more busses and in computer communication with one another. Similarly, components of the system 100 of FIG. 1 may be communicatively coupled via a network and/or wireless communication and be in computer communication with one another, for example.

### Input Dataset for Model Generation

At a high level, the model generator 160 may be utilized to generate an activity prediction model 152 based on the input dataset 170 and using machine learning. The input dataset 170 may include a set of sensor data associated with activity of an individual from a training phase and a set of flags associated with the set of sensor data. This set of flags may be considered as ground truth for the machine learning training indicative of a posture of the individual and an onset, start, or beginning of a transition or a change from a first activity state to a second activity state for that individual. In this way, the input dataset may include the set of sensor data associated with activity of the individual from the training phase and a set of flags associated with the set of sensor data. One or more of the flags from the set of flags may be indicative of an onset of a transition from a first activity state to a second activity state for the individual from the training phase. Explained another way, a portion of the set of sensor data of the input dataset 170 may be viewed as a ‘question’ and the set of flags may be viewed as an ‘answer’ (e.g., yes, this person is about to get up) to the ‘question’ (e.g., is this person about to get up?). Providing the model generator 160 with this input dataset 170 may thus facilitate the building of the activity prediction model 152. Additionally, it is generally desirable to produce an activity prediction model 152 which is fitted to the input dataset 170 and generalizes well to new, unknown data, as will be described herein.

According to one aspect, the sensor data may be accelerometer data which is received while the individual is engaged in a variety of movements or activities. Here, assuming the sensor type is the accelerometer, the accelerometer may be affixed to the individual's chest or other parts of the body, such as wrists, legs, arms, etc. Moreover, one or more of the sensors may be affixed to the individual. Examples of activities may include lying down, sitting, walking, standing, reaching for objects, going to the restroom, eating, showering, brushing teeth, getting dressed, preparing meals, performing chores, walking pets, commuting, bicycling, playing with children, engaging in sporting activities, etc. As the individual engages in the variety of activities, any number of transitions may occur between these respective activities. For example, the individual may go from sitting to laying down or from laying down to sitting. As additional examples, the individual may engage in walking to sitting, walking to laying down, standing to laying down, standing to sitting, standing to walking, sitting to standing, sitting to walking, laying down to walking, laying down to standing, etc. Assuming the sensor type is the accelerometer, the accelerometer may sense or detect accelerometer data from the activity of the individual. With respect to the training phase, the accelerometer data may be transmitted (e.g., wirelessly transmitted or via a wired connection when the accelerometer is taken off and plugged into a computer) to the communication interface 166 of the model generator 160. With respect to the execution phase, the accelerometer data may be transmitted by the transmitter 112 of the sensor 110 to the communication interface 130 of the mobile device 120, which may then pass the accelerometer data to the trained model 152, according to one aspect.

As previously discussed, the input dataset 170 may include the set of flags associated with the set of sensor data. This set of flags may be provided via human annotation or provided by model annotation (e.g., unsupervised machine learning) and represent the ground truth as to the posture of the individual or transitions between postures. For example, participants may be observed by sensors doing everyday activities, and these activities may be annotated (e.g., by human or machine model) after the fact. Based on the data received from the sensors during the training phase, the prediction model may be trained and utilized to make predictions with regard to activity of an individual.

According to one aspect, the flags may be placed an onset, a beginning, or a start of a transition or the change from one activity state to another activity state. For example, a human annotator may direct a participant who is being observed by the sensors in associated with generation of the input dataset 170 to perform different activities, such as going from walking to sitting. It should be noted that the input dataset 170 is not necessarily required to be derived from participants who have been directed to perform specific actions. The human annotator may then (during the collection of the input dataset 170 or anytime thereafter) manually flag portions of each activity state, such as the onset of the walking, completion of the walking, the onset of the sitting, steady state sitting, etc. According to another aspect a machine model may make these annotations. In any event, the set of flags may be indicative of a transition or a change from a first activity state to a second activity state or an onset of the transition or the change from the first activity state to the second activity state. According to one aspect, the individual may be defined to be in one of three activity states: an inactive activity state, a sitting activity state, or an active activity state. However, it will be appreciated that any number of activity states may be utilized.

As discussed, the set of flags may be indicative of a transition from a first activity state to a second activity state. For example, a flag from the set of flags may be an annotation indicating when the individual engages in a sitting to standing activity. Stated another way, the flag may be an annotation indicating when the individual is about to go (e.g., onset of transition) from a sitting activity to a standing activity. Therefore, using this input dataset 170, the activity prediction model 152 may be trained to predict when an individual is about to transition from one activity posture to another activity posture. Stated again, the activity prediction model 152, which is trained using the input dataset 170 may predict or estimate an onset of a transition or a change from a first activity state to a second activity state prior to an individual actually engaging in transitioning between the activity states. In this way, the activity prediction model 152 may be built or trained to more accurately predict when an individual will transition from a first activity state to a second activity state, and thus, predict an ‘intention’ of the individual.

Explained another way, flags may be annotations indicative of a transition from a lower activity posture to a higher activity posture or indicative of a transition from a higher activity posture to a lower activity posture. It should be noted that the transition may not necessarily begin precisely at the onset of movement. In other words, the transition may include a brief period of time, activity, or postures where the individual is about to change activities and has not yet initiated any movement. In this way, the flags may be indicative of an onset of transitioning from one activity posture to another. For the purpose of discussion, and by way of example, the inactive activity state may be considered a lower activity posture, the sitting activity state as a higher activity posture than the inactive activity state, and the active activity state as the highest activity posture, although fewer or additional activity states may be utilized in other embodiments. Therefore, flags may be indicative of transitions between states and may clearly identify whether the individual is in the inactive activity state, the sitting activity state, or the active activity state.

According to one aspect, the inactive activity state may include laying down. The sitting activity state may include sitting, sitting to laying down, and laying down to sitting. The active activity state may include walking, walking to sitting, walking to laying down, standing, standing to laying down, standing to sitting, standing to walking, sitting to standing, sitting to walking, laying to walking, and laying to standing.

Further, although the input dataset 170 is discussed in terms of a set of accelerometer sensor data, it will be appreciated that any type and/or combination of sensors may be utilized for the input dataset 170. For example, the input dataset 170 may include a set of sensor data from one or more of an accelerometer, an image capture device, a video device, a heartbeat sensor, a cardiac sensor, a temperature sensor, an electrocardiogram sensor, an oxygen sensor, a blood pressure sensor, a biometric sensor, a non-contact sensor, such as Lidar, radar, infrared, any other sensor, etc. Regardless, the input dataset 170 may be received by the communication interface 166 of the model generator 160. In this way, the model generator 160 may generate the activity prediction model 152 based on this input dataset 170.

### Model Generation

According to one aspect, the model generator 160 may generate, during a model training phase, via the processor 162 and memory 164, the activity prediction model 152 based on the input dataset 170 (which includes known data or ground truth data) using machine learning to teach the activity prediction model 152 associations between the set of sensor data and the set of flags. Once the model has learned these associations, the model may be utilized to generate new predictions on additional or new data. If the model is wrong, adjustments may be made by the model generator 160 and additional rounds of training may be provided. This may be achieved using a variety of machine learning data partitioning techniques or strategies, including but not limited to train-test-validate, train-validate-test, test-validate-train, test-train, train-test, leave one out, etc. The training for the model generator 160 may be handled using an external library.

The input dataset 170 may be divided into two or more subsets, depending on the machine learning technique utilized or selected. In other words, the input dataset 170 may be partitioned or divided into two or more subsets (e.g., a first subset, a second subset, a third subset, etc.). Each subset may be of a type from one of a training data subset, a validation data subset, and a test data subset. For the sake of simplicity, discussion of model generation herein will be provided assuming the train-test-validate technique, although as discussed above, any of a wide variety of techniques may be implemented. In this regard, with reference to the train-test-validate technique, the input dataset 170 may be divided into three subsets (e.g., the training data subset, the validation data subset, and the test data subset). It should be noted that overlap between data of the subsets may be permitted or possible according to some variations or aspects.

In any event, the processor 162 of the model generator 160 may train two or more potential activity prediction models using machine learning based on a first subset of the input dataset 170 and one or more hyperparameters. The initial training may be performed using randomly defined hyperparameters while subsequent training may be done with variations to the initial hyperparameters based on evaluated performance. With reference to the train-test-validate technique, the two or more potential activity prediction models may be trained based on the training data subset as the first subset of the input dataset 170. According to one aspect, the training of the two or more potential activity prediction models may be performed using different hyperparameters for each of the two or more potential activity prediction models, as will be discussed in greater detail herein.

Training data (e.g., the data from the training data subset) may be a dataset used by the model generator 160 to train the two or more potential activity prediction models and may be used to fit parameters, such as weights, of a classifier for a given potential activity prediction model. Generally, for classification, a supervised learning algorithm of the model generator 160 may examine the training data set to determine, or to learn, optimal combinations of variables that may be utilized to generate an accurate predictive model (i.e., the potential activity prediction models). Although the supervised learning algorithm is described above, it will be appreciated that unsupervised machine learning may be implemented. For example, machine learning algorithms may analyze and cluster unlabeled data sets as the training data set to determine, or to learn, optimal combinations of variables that may be utilized to generate an accurate predictive model.

The processor 162 of the model generator 160 may optimize one or more of the hyperparameters of the two or more potential activity prediction models based on a second subset of the input dataset 170. Here, with reference to the train-test-validate technique, the second subset of the input dataset 170 may be the validation data subset. Explained another way, the processor 162 of the model generator 160 may optimize one or more of the hyperparameters to fit the validation data subset.

Validation data (e.g., the data from the validation data subset) may be a dataset used to tune the hyperparameters (e.g., the architecture) of the aforementioned classifier for a given potential activity prediction model or to test the model to find the best set of hyperparameters. According to one aspect, Bayesian optimization may be implemented for hyperparameter optimization and/or validation.

A hyperparameter may be a parameter whose value is used to control the learning process for the potential activity prediction models. Examples of hyperparameters may include, but are not limited to a type of neural network utilized (e.g., a convolutional neural network (CNN), a recurrent neural network (RNN), or a long short-term memory (LSTM) neural network), a number of convolutions, a size of convolutions, forgetfulness, a number of layers for a given neural network, a number of neurons, a number of hidden units in each layer, featurization, or a window size associated with the set of sensor data for a given prediction, etc.

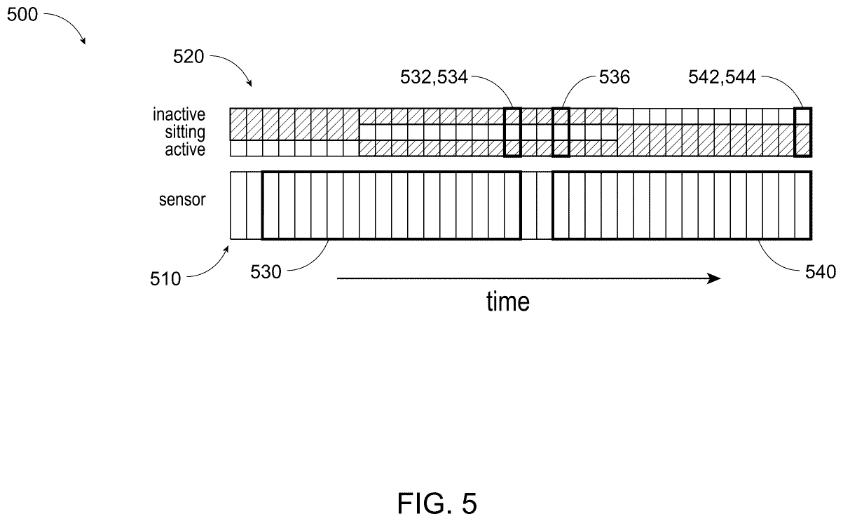

The processor 162 of the model generator 160 may perform one or more of the training potential activity prediction models, optimizing the hyperparameters, or other aspects of model generation by adjusting the window size associated with the set of sensor data with reference to an activity prediction. As discussed above, a portion of the set of sensor data of the input dataset 170 may be viewed as a ‘question’ (e.g., what is the posture of the individual currently?) and the set of flags may be viewed as an ‘answer’ (e.g., laying down) to the ‘question’. However, to train the activity prediction model 152, a certain amount of data may be provided as an input to the model. In this regard, the window size associated with the set of sensor data may be a predetermined number of time steps from the set of sensor data which may be utilized to generate the activity prediction and indicative of the amount of data which is to be provided as the input to the model during the training phase to train the activity prediction model 152. During the execution phase, the same or a similar window size of sensor data may be provided to obtain the activity prediction from the activity prediction model 152. An example of this window may be seen in FIG. 5, which will be discussed in greater detail herein.

The processor 162 of the model generator 160 may select one of the two or more potential activity prediction models as the activity prediction model 152 based on a third subset of the input dataset 170. Here, with reference to the train-test-validate technique, the third subset of the input dataset 170 may be the test data subset. Further, according to the train-test-validate technique, the optimizing one or more of the hyperparameters may occur prior to the selecting one of the two or more potential activity prediction models as the activity prediction model 152. Alternatively, if the train-validate-test technique is utilized, the selecting one of the two or more potential activity prediction models as the activity prediction model 152 may occur prior to the optimizing one or more of the hyperparameters.

Test data (e.g., the data from the test data subset) may be a dataset which is independent from the training data subset and may be utilized to assess the performance (e.g., generalization) of a given classifier. In other words, the test data or the test data subset may be utilized to generally generate classification predictions from the two or more potential activity prediction models. One specific type of classification prediction may be an activity prediction. These classification predictions or activity predictions may be compared to known ground truth classifications (e.g., the set of flags) to assess model accuracy for the respective potential activity prediction models.

### Activity Prediction Model Training

In this way, the processor 162 of the model generator 160 may generate the activity prediction model 152 which may be a machine learning model or a function y=f(x) which maps an input x to an output y. This activity prediction model 152 may be transmitted to the server 150 for hosting and/or storage for use during the execution phase of activity prediction. According to an alternate aspect, the mobile device 120 may host the activity prediction model 152.

In any event, the activity prediction model 152 may generate an output y of an activity prediction (i.e., a specific type of classification prediction) based on an execution dataset (from the sensor 110) including an execution set of sensor data associated with activity of an individual from an execution phase. The activity prediction y may be from a set of two or more activity prediction states, such as the inactive activity state, the sitting activity state, or the active activity state. According to one aspect, a window of time steps (which may be a predetermined number of time steps) of sensor data from the execution dataset may be utilized as input x to the activity prediction model 152 f. As previously discussed, this input x to the activity prediction model 152 may include a set of sensor data (e.g., accelerometer data) associated with activity of an individual over a period of time known as the time window.

The activity prediction may be indicative of a current activity of the individual or a future activity of the individual, depending on how the window associated with the set of sensor data is setup for a given prediction and how the activity prediction model was trained during the training phase. Additionally, the activity prediction may be indicative of a transition from a lower activity posture to a higher activity posture or may be indicative of a transition from a higher activity posture to a lower activity posture for the individual. For example, the activity prediction may be a prediction made by the activity prediction model 152 from the execution set of sensor data, such as from an accelerometer, that the individual associated with the execution set of sensor data is currently transitioning from the sitting activity state to the active activity state.

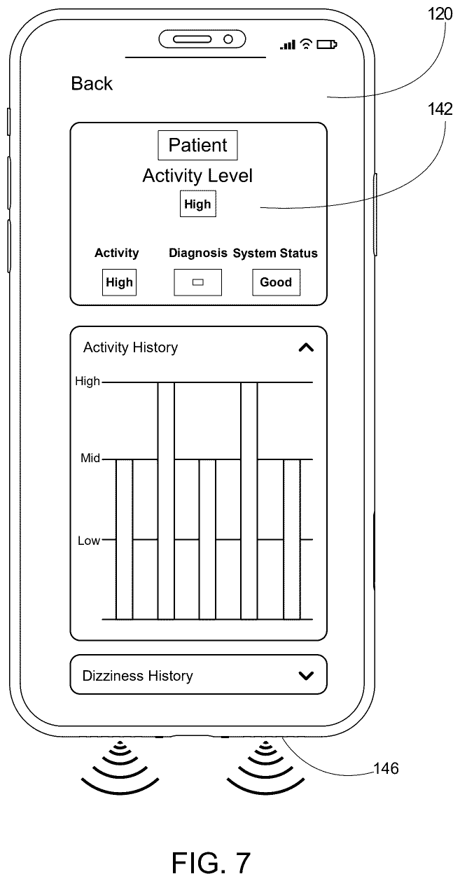

The activity prediction may be transmitted from the server 150 to the receiver 134 of the communication interface 130 of the mobile device 120. Thereafter, the application 142 executed on the mobile device 120 may analyze the activity prediction to determine whether the activity prediction is associated with a risk of injury due to an anticipated movement or activity, such as a chance of falling greater than a threshold amount, such as a confidence level that the individual is in the active activity state. Other transformations may be applied to the activity prediction to determine whether or not to provide a notification. If the activity prediction is associated with a risk of injury due to an anticipated movement or activity or a chance of falling greater than a threshold amount, the application 142 may cause the display 144 and/or the speaker 146 of the mobile device 120 to generate the notification, as will be described below with reference to FIG. 7.

Similarly, to the input dataset 170, while the execution dataset is discussed herein in terms of a set of accelerometer sensor data, it will be appreciated that any type and/or combination of sensors may be utilized for the execution dataset. For example, the execution dataset may include a set of sensor data from one or more of an accelerometer, an image capture device, a heartbeat sensor, a cardiac sensor, a temperature sensor, an electrocardiogram sensor, an oxygen sensor, a blood pressure sensor, a biometric sensor, any other sensor, etc. and be associated with activity of an individual. The execution dataset may be received by the receiver 134 of the communication interface 130 of the mobile device 120 which may pass the execution dataset to the activity prediction model 152 hosted by the server 150. Alternatively, the transmitter 112 on the sensor 110 may transmit the execution dataset directly to the activity prediction model 152 hosted by the server 150, which may generate the activity prediction based thereon. In any event, the processor of the device hosting the activity prediction model 152 (e.g., server 150 in FIG. 1 or the processor 122 of the mobile device 120 according to another embodiment) may pass the execution dataset through the activity prediction model 152 to generate the activity prediction.

### Train-Test-Validate

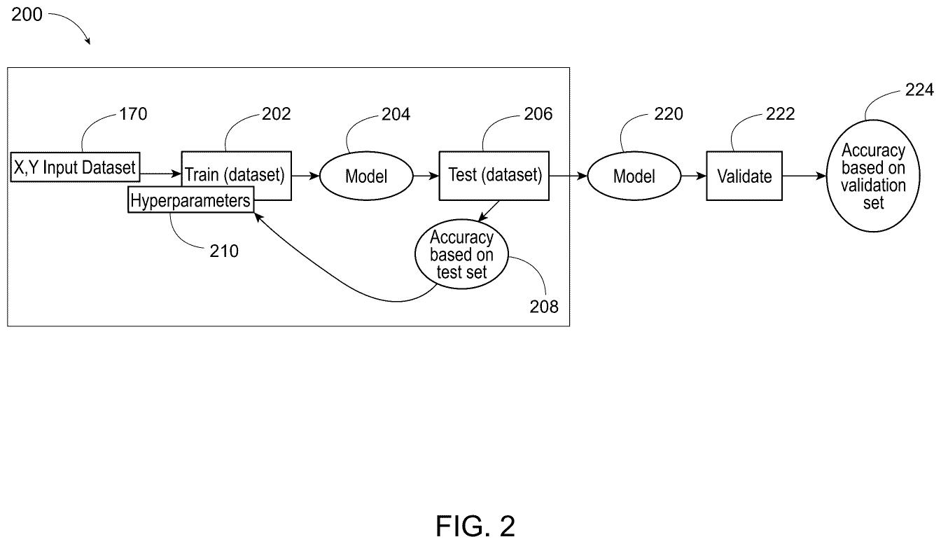

FIG. 2 is an exemplary flow diagram of a method 200 for training an activity prediction model, according to one aspect. The model generator 160 may perform training of the model iteratively. For example, processor 162 of the model generator 160 may train 202 potential activity prediction models 204 using machine learning based on the input dataset 170 (e.g., including the training data subset) and a set of one or more hyperparameters 210.

Thereafter, the potential activity prediction models may be evaluated 206 or tested using the test data subset. Based on the evaluation 208, a similar set of hyperparameters may be selected and/or utilized to train additional potential activity prediction models or a dissimilar or different set of hyperparameters may be utilized to train additional potential activity prediction models 220. In this way, optimization of hyperparameters 210 to fit to the test data subset may occur. Validation 222 may be performed to determine accuracy 224, such as whether the potential activity prediction models 220 are overfit to the test data subset. In this way, hyperparameter optimization is optimized to the test data subset rather than the validation data subset, according to this aspect.

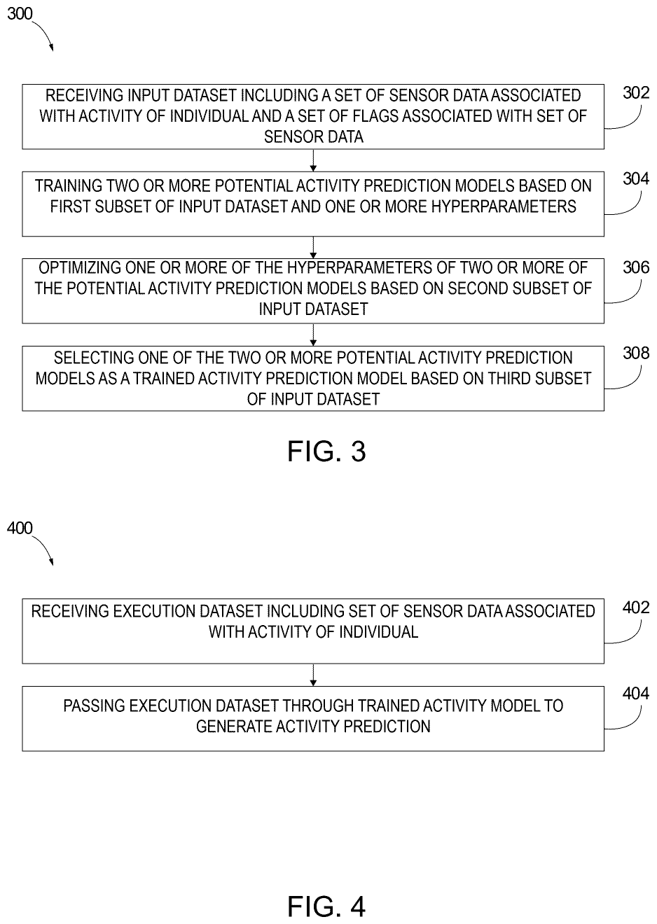

FIG. 3 is an exemplary flow diagram of a method 300 for training an activity prediction model, according to one aspect. The method 300 for training the activity prediction model may include receiving 302 the input dataset 170 including a set of sensor data associated with activity of an individual and a set of flags associated with the set of sensor data, training 304 two or more potential activity prediction models using machine learning based on a first subset of the input dataset 170 and one or more hyperparameters, optimizing 306 one or more of the hyperparameters of the two or more potential activity prediction models based on a second subset of the input dataset 170, and selecting 308 one of the two or more potential activity prediction models as the activity prediction model 152 based on a third subset of the input dataset 170.

FIG. 4 is an exemplary flow diagram of a method 400 for activity prediction, according to one aspect. The method 400 for activity prediction may include receiving 402 an execution dataset including a set of sensor data associated with activity of an individual and passing 404 the execution dataset through the activity prediction model 152 to generate an activity prediction. Once the activity prediction is generated, an alert may be generated on a corresponding mobile device if desired. The alert may be provided if the activity prediction is associated with a risk of injury due to an anticipated movement or activity, such as a chance of falling greater than a threshold amount, for example.

FIG. 5 is an exemplary illustration of data 500 which may be utilized in accordance with any of the systems or methods of FIGS. 1-4, according to one aspect. The data 500 may be from the input dataset 170 or from the execution dataset.

According to one aspect, assuming that the data 500 of FIG. 5 is from the input dataset 170, a set of sensor data 510 may be associated with activity of the individual and a set of flags 520 may be associated with the set of sensor data 510. Here, a first window of sensor data 530 may be associated with a first flag 532 (e.g., going from the active activity state to the sitting activity state) and a second window of sensor data 540 may be associated with a second flag 542 (e.g., going from the sitting activity state to the inactive activity state). According to one aspect, during the training phase, the model generator 160 may train the activity prediction model 152 to predict the future activity 536 of the individual based on the first window of sensor data 530 provided.

According to another aspect, assuming that the data 500 of FIG. 5 is from the execution dataset, 510 may be the set of sensor data associated with activity of the individual and 520 may be the output or activity prediction generated according to the activity prediction model 152. Here, the activity prediction model 152 may receive the first window of sensor data 530 and generate an activity prediction 534 (e.g., going from the active activity state to the sitting activity state) and receive the second window of sensor data 540 and generate an activity prediction 544 (e.g., going from the sitting activity state to the inactive activity state). According to one aspect, during the execution phase, the model generator 160 may predict future activity 536 of the individual based on the first window of sensor data 530 provided rather than the current activity at the activity prediction 534.

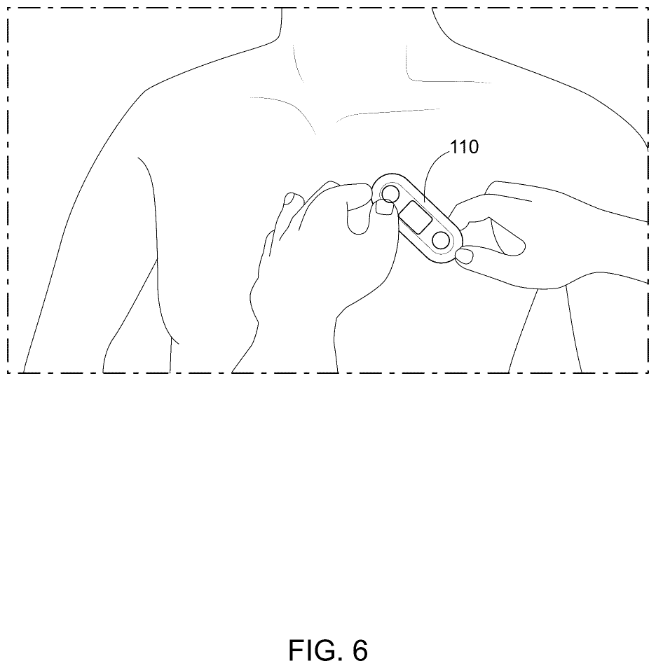

FIG. 6 is an exemplary sensor for the system 100 for activity prediction of FIG. 1, according to one aspect. As previously discussed, the sensor 110 may be an accelerometer, an image capture device, a video device, a heartbeat sensor, a cardiac sensor, a temperature sensor, an electrocardiogram sensor, an oxygen sensor, a blood pressure sensor, a biometric sensor, a non-contact sensor, such as Lidar, radar, infrared, etc. As seen in FIG. 6, the sensor 110 may be affixed or mounted to a patient or individual for data gathering or collection.

FIG. 7 is an exemplary application 142 which may be executed on the mobile device 120 of the system 100 for activity prediction of FIG. 1, according to one aspect. In FIG. 7, when the activity prediction model 152 generates the activity prediction and transmits the activity prediction to the communication interface 130 of the mobile device 120, if the activity prediction is associated with a risk of injury due to an anticipated movement or activity, an activity level greater than a threshold amount, a chance of falling greater than a threshold amount, the application 142 of the mobile device 120 may generate one or more notifications which may be provided to a caregiver, for example. Although the notification provided in this example is an audible alarm via the speaker 146 and a display notification on the display 144, any other type of notification may be implemented.

The following includes definitions of selected terms employed herein. The definitions include various examples and/or forms of components that fall within the scope of a term and that may be used for implementation. The examples are not intended to be limiting. Further, one having ordinary skill in the art will appreciate that the components discussed herein, may be combined, omitted, or organized with other components or organized into different architectures.

A “processor”, as used herein, processes signals and performs general computing and arithmetic functions. Signals processed by the processor may include digital signals, data signals, computer instructions, processor instructions, messages, a bit, a bit stream, or other means that may be received, transmitted, and/or detected. Generally, the processor may be a variety of various processors including multiple single and multicore processors and co-processors and other multiple single and multicore processor and co-processor architectures. The processor may include various modules to execute various functions.

A “memory”, as used herein, may include volatile memory and/or non-volatile memory. Non-volatile memory may include, for example, ROM (read only memory), PROM (programmable read only memory), EPROM (erasable PROM), and EEPROM (electrically erasable PROM). Volatile memory may include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory may store an operating system that controls or allocates resources of a computing device.

A “disk” or “drive”, as used herein, may be a magnetic disk drive, a solid state disk drive, a floppy disk drive, a tape drive, a Zip drive, a flash memory card, and/or a memory stick. Furthermore, the disk may be a CD-ROM (compact disk ROM), a CD recordable drive (CD-R drive), a CD rewritable drive (CD-RW drive), and/or a digital video ROM drive (DVD-ROM). The disk may store an operating system that controls or allocates resources of a computing device.

A “bus”, as used herein, refers to an interconnected architecture that is operably connected to other computer components inside a computer or between computers. The bus may transfer data between the computer components. The bus may be a memory bus, a memory controller, a peripheral bus, an external bus, a crossbar switch, and/or a local bus, among others.

A “database”, as used herein, may refer to a table, a set of tables, and a set of data stores (e.g., disks) and/or methods for accessing and/or manipulating those data stores.

An “operable connection”, or a connection by which entities are “operably connected”, is one in which signals, physical communications, and/or logical communications may be sent and/or received. An operable connection may include a wireless interface, a physical interface, a data interface, and/or an electrical interface.

A “computer communication”, as used herein, refers to a communication between two or more computing devices (e.g., computer, personal digital assistant, cellular telephone, network device) and may be, for example, a network transfer, a file transfer, an applet transfer, an email, a hypertext transfer protocol (HTTP) transfer, and so on. A computer communication may occur across, for example, a wireless system (e.g., IEEE 802.11), an Ethernet system (e.g., IEEE 802.3), a token ring system (e.g., IEEE 802.5), a local area network (LAN), a wide area network (WAN), a point-to-point system, a circuit switching system, a packet switching system, among others.

A “mobile device”, as used herein, may be a computing device typically having a display screen with a user input (e.g., touch, keyboard) and a processor for computing. Mobile devices include handheld devices, portable electronic devices, smart phones, laptops, tablets, and e-readers.

The aspects discussed herein may be described and implemented in the context of non-transitory computer-readable storage medium storing computer-executable instructions. Non-transitory computer-readable storage media include computer storage media and communication media. For example, flash memory drives, digital versatile discs (DVDs), compact discs (CDs), floppy disks, and tape cassettes. Non-transitory computer-readable storage media may include volatile and non-volatile, removable and non-removable media implemented in any method or technology for storage of information such as computer readable instructions, data structures, modules, or other data.

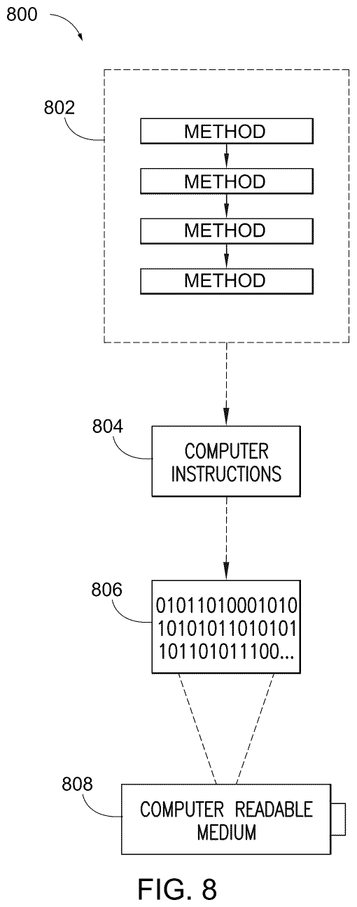

Still another aspect involves a computer-readable medium including processor-executable instructions configured to implement one aspect of the techniques presented herein. An aspect of a computer-readable medium or a computer-readable device devised in these ways is illustrated in FIG. 8, wherein an implementation 800 includes a computer-readable medium 808, such as a CD-R, DVD-R, flash drive, a platter of a hard disk drive, etc., on which is encoded computer-readable data 806. This encoded computer-readable data 806, such as binary data including a plurality of zero's and one's as shown in 806, in turn includes a set of processor-executable computer instructions 804 configured to operate according to one or more of the principles set forth herein. In this implementation 800, the processor-executable computer instructions 804 may be configured to perform a method 802, such as the method 200 of FIG. 2, the method 300 of FIG. 3, the method 400 of FIG. 4. In another aspect, the processor-executable computer instructions 804 may be configured to implement a system, such as the system 100 of FIG. 1. Many such computer-readable media may be devised by those of ordinary skill in the art that are configured to operate in accordance with the techniques presented herein.

As used in this application, the terms “component”, “module,” “system”, “interface”, and the like are generally intended to refer to a computer-related entity, either hardware, a combination of hardware and software, software, or software in execution. For example, a component may be, but is not limited to being, a process running on a processor, a processing unit, an object, an executable, a thread of execution, a program, or a computer. By way of illustration, both an application running on a controller and the controller may be a component. One or more components residing within a process or thread of execution and a component may be localized on one computer or distributed between two or more computers.

Further, the claimed subject matter is implemented as a method, apparatus, or article of manufacture using standard programming or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

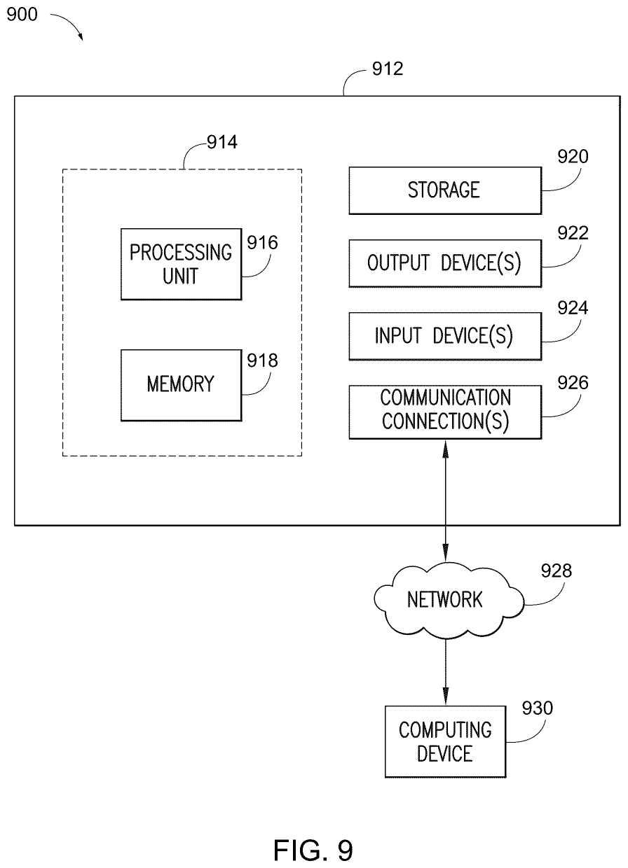

FIG. 9 and the following discussion provide a description of a suitable computing environment to implement aspects of one or more of the provisions set forth herein. The operating environment of FIG. 9 is merely one example of a suitable operating environment and is not intended to suggest any limitation as to the scope of use or functionality of the operating environment. Example computing devices include, but are not limited to, personal computers, server computers, hand-held or laptop devices, mobile devices, such as mobile phones, Personal Digital Assistants (PDAs), media players, and the like, multiprocessor systems, consumer electronics, mini computers, mainframe computers, distributed computing environments that include any of the above systems or devices, etc.

Generally, aspects are described in the general context of “computer readable instructions” being executed by one or more computing devices. Computer readable instructions may be distributed via computer readable media as will be discussed below. Computer readable instructions may be implemented as program modules, such as functions, objects, Application Programming Interfaces (APIs), data structures, and the like, that perform one or more tasks or implement one or more abstract data types. Typically, the functionality of the computer readable instructions are combined or distributed as desired in various environments.

FIG. 9 illustrates a system 900 including a computing device 912 configured to implement one aspect provided herein. In one configuration, the computing device 912 includes at least one processing unit 916 and memory 918. Depending on the exact configuration and type of computing device, memory 918 may be volatile, such as RAM, non-volatile, such as ROM, flash memory, etc., or a combination of the two. This configuration is illustrated in FIG. 9 by dashed line 914.

In other aspects, the computing device 912 includes additional features or functionality. For example, the computing device 912 may include additional storage such as removable storage or non-removable storage, including, but not limited to, magnetic storage, optical storage, etc. Such additional storage is illustrated in FIG. 9 by storage 920. In one aspect, computer readable instructions to implement one aspect provided herein are in storage 920. Storage 920 may store other computer readable instructions to implement an operating system, an application program, etc. Computer readable instructions may be loaded in memory 918 for execution by the at least one processing unit 916, for example.

The term “computer readable media” as used herein includes computer storage media. Computer storage media includes volatile and nonvolatile, removable, and non-removable media implemented in any method or technology for storage of information such as computer readable instructions or other data. Memory 918 and storage 920 are examples of computer storage media. Computer storage media includes, but is not limited to, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which may be used to store the desired information and which may be accessed by the computing device 912. Any such computer storage media is part of the computing device 912.

The term “computer readable media” includes communication media. Communication media typically embodies computer readable instructions or other data in a “modulated data signal” such as a carrier wave or other transport mechanism and includes any information delivery media. The term “modulated data signal” includes a signal that has one or more of its characteristics set or changed in such a manner as to encode information in the signal.

The computing device 912 includes input device(s) 924 such as keyboard, mouse, pen, voice input device, touch input device, infrared cameras, video input devices, or any other input device. Output device(s) 922 such as one or more displays, speakers, printers, or any other output device may be included with the computing device 912. Input device(s) 924 and output device(s) 922 may be connected to the computing device 912 via a wired connection, wireless connection, or any combination thereof. In one aspect, an input device or an output device from another computing device may be used as input device(s) 924 or output device(s) 922 for the computing device 912. The computing device 912 may include communication connection(s) 926 to facilitate communications with one or more other devices 930, such as through network 928, for example.

Although the subject matter has been described in language specific to structural features or methodological acts, it is to be understood that the subject matter of the appended claims is not necessarily limited to the specific features or acts described above. Rather, the specific features and acts described above are disclosed as example aspects.

Various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

As used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Further, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel. Additionally, “comprising”, “comprises”, “including”, “includes”, or the like generally means comprising or including, but not limited to.

It will be appreciated that various of the above-disclosed and other features and functions, or alternatives or varieties thereof, may be desirably combined into many other different systems or applications. Also, that various presently unforeseen or unanticipated alternatives, modifications, variations, or improvements therein may be subsequently made by those skilled in the art which are also intended to be encompassed by the following claims.

1. A system for training an activity prediction model, comprising:
a memory storing one or more instructions;
a processor executing one or more of the instructions stored on the memory to perform:
receiving an input dataset, wherein the input dataset includes a set of sensor data associated with activity of an individual from a training phase and a set of flags associated with the set of sensor data, wherein one or more of the flags from the set of flags is indicative of an onset of a transition from a first activity state to a second activity state for the individual from the training phase;
training two or more potential activity prediction models using machine learning based on a first subset of the input dataset and one or more hyperparameters;
optimizing one or more of the hyperparameters of the two or more potential activity prediction models based on a second subset of the input dataset; and
selecting one of the two or more potential activity prediction models as an activity prediction model based on a third subset of the input dataset,
wherein the activity prediction model generates an activity prediction based on an execution dataset including a second set of sensor data associated with activity of a second individual during an execution phase.

2. The system for training an activity prediction model of claim 1, wherein the set of sensor data includes accelerometer data associated with activity of the individual.

3. The system for training an activity prediction model of claim 1, wherein the set of flags associated with the set of sensor data is human annotated or model annotated.

4. The system for training an activity prediction model of claim 1, wherein the optimizing one or more of the hyperparameters occurs prior to the selecting one of the two or more potential activity prediction models as the activity prediction model.

5. The system for training an activity prediction model of claim 1, wherein the selecting one of the two or more potential activity prediction models as the activity prediction model occurs prior to the optimizing one or more of the hyperparameters.

6. The system for training an activity prediction model of claim 1, wherein one or more of the hyperparameters includes a type of neural network, a number of convolutions, a number of layers, or a window size associated with the set of sensor data.

7. The system for training an activity prediction model of claim 6, wherein the type of neural network includes a convolutional neural network (CNN), a recurrent neural network (RNN), or a long short-term memory (LSTM) neural network.

8. The system for training an activity prediction model of claim 1, wherein the activity prediction generated by the activity prediction model is indicative of a current activity of the second individual.

9. The system for training an activity prediction model of claim 1, wherein the activity prediction generated by the activity prediction model is indicative of a future activity of the second individual.

10. The system for training an activity prediction model of claim 1, wherein the training of the two or more potential activity prediction models is performed using different hyperparameters for each of the two or more potential activity prediction models.

11. A system for activity prediction, comprising:
a memory storing one or more instructions;
a processor executing one or more of the instructions stored on the memory to perform:
receiving an execution dataset, wherein the execution dataset includes a set of sensor data associated with activity of an individual from an execution phase; and
passing the execution dataset through an activity prediction model to generate an activity prediction,
wherein the activity prediction model is trained using machine learning based on a first subset of an input dataset and one or more hyperparameters,
wherein the input dataset includes a set of sensor data associated with activity of an individual from a training phase and a set of flags associated with the set of sensor data, wherein one or more of the flags from the set of flags is indicative of an onset of a transition from a first activity state to a second activity state for the individual from the training phase,
wherein one or more of the hyperparameters is optimized based on a second subset of the input dataset,
wherein the activity prediction model is selected from two or more potential activity prediction models based on a third subset of the input dataset.

12. The system for activity prediction of claim 11, comprising a sensor sensing data of the execution dataset and transmitting the data to the processor.

13. The system for activity prediction of claim 12, wherein the sensor is an accelerometer, an image capture device, a heartbeat sensor, a cardiac sensor, a temperature sensor, an electrocardiogram sensor, an oxygen sensor, a blood pressure sensor, or a biometric sensor.

14. The system for activity prediction of claim 11, wherein one or more of the hyperparameters includes a type of neural network, a number of convolutions, a number of layers, or a window size associated with the set of sensor data.

15. The system for activity prediction of claim 11, wherein the activity prediction generated by the activity prediction model is indicative of a current activity of the individual from the execution phase.

16. The system for activity prediction of claim 11, wherein the activity prediction generated by the activity prediction model is indicative of a future activity of the individual from the execution phase.

17. A computer-implemented method for activity prediction, comprising:
receiving, via a processor, an execution dataset, wherein the execution dataset includes a set of sensor data associated with activity of an individual from an execution phase; and
passing, via the processor, the execution dataset through an activity prediction model to generate an activity prediction,
wherein the activity prediction model is trained using machine learning based on a first subset of an input dataset and one or more hyperparameters,
wherein the input dataset includes a set of sensor data associated with activity of an individual from a training phase and a set of flags associated with the set of sensor data, wherein one or more of the flags from the set of flags is indicative of an onset of a transition from a first activity state to a second activity state for the individual from the training phase,
wherein one or more of the hyperparameters is optimized based on a second subset of the input dataset,
wherein the activity prediction model is selected from two or more potential activity prediction models based on a third subset of the input dataset.

18. The computer-implemented method for activity prediction of claim 17, wherein the first activity state from the input dataset is laying down and the second activity state from the input dataset is a higher activity posture than the first activity state of laying down.

19. The computer-implemented method for activity prediction of claim 17, wherein the sensor is an accelerometer, an image capture device, a heartbeat sensor, a cardiac sensor, a temperature sensor, an electrocardiogram sensor, an oxygen sensor, a blood pressure sensor, or a biometric sensor.

20. The computer-implemented method for activity prediction of claim 17, wherein one or more of the hyperparameters includes a type of neural network, a number of convolutions, a number of layers, or a window size associated with the set of sensor data.
